(19) United States
(12) Patent Application Publication
(10) Pub. No.: US 2007/0293945 A1
(43) Pub. Date: Dec. 20, 2007

(54) LIGHTWEIGHT BREAST PROSTHESIS

(76) Inventor: Johnny Snyder, China Spring, TX (US)

Correspondence Address: MARCUS HAMMACK;NAMAN, HOWELL, SMITH & LEE, P.O. BOX 1470, 900 WASHINGTON, WACO, TX, 76703, US

(21) Appl. No.: 11/423,972

(22) Filed: Jun. 14, 2006

(51) Int. Cl. A61F 2/52

(52) U.S. Cl. 623/7

(57) ABSTRACT

The present invention is directed toward a lightweight, external breast prosthesis. In particular, the present invention provides a breast prosthesis comprised of three material layers which allow the production of an extremely light prosthesis while maintaining the look, shape, feel, and behavior of a natural human breast. The outermost layer is comprised of reduced density gel formed in the shape of a human breast. The second layer is a thin layer of standard density gel comprised to provide support and firmness to the outer layer. The innermost layer is a removable foam insert with a sticky coating enabling the innermost layer to “grab” the second layer allowing the three layers of the prosthesis to move as a single unit while maintaining the look, feel, and behavior of a natural human breast at a fraction of the weight of a standard silicone breast prosthesis.

### BACKGROUND OF THE INVENTION

1. Field of the Invention

The present invention is directed toward lightweight breast prostheses. In particular, the present invention provides a breast prosthesis comprised of three material layers which allow the production of an extremely light prosthesis while maintaining the look, shape, feel, and behavior of a natural human breast.

2. Background Information

Surgical breast implants have been available for a number of years to provide patients with the most realistic look and feel of a natural breast; however, this option is not only wrought with all of the risks of the surgical procedure itself, but also, with potential complications with the implanted device throughout the life of the patient. Consequently, over the years, a number of external breast prostheses have been developed in an attempt to provide mastectomy patients with the most realistic look, feel, and behavior possible without the associated risk and stress of surgery. Moreover, it is essential for the psychological well-being of the patient that the prosthesis mimics the natural human breast such that its presence will not be detected by others in the ordinary course of the patient's activities. However, not only must the prosthesis look and feel natural, it must also be comfortable enough for the patient to wear throughout her waking hours.

Generally, silicone gel has become the most widely accepted material used in external breast prostheses, for the most part because of its resilient properties. A prime example of this style of external breast prosthesis is disclosed in U.S. Pat. No. 4,019,209 issued to Spence. However, in the ensuing years it became apparent that the weight of a breast prosthesis composed entirely of silicone gel is such that it produces undo strain on the mastectomy patient's back, resulting in side effects ranging from discomfort to painful back injuries. As a result, significant effort has been undertaken to develop a lighter weight breast prosthesis without losing the look, feel and behavior of a natural breast. As a result of U.S. Pat. No. 4,380,569 by Shaw, the lightweight breast prosthesis industry moved toward a lightweight, preformed gel composition comprised of a mixture of silicon gel and glass microspheres. This product and method enabled production of a lighter weight breast prosthesis, however, at the expense of the natural look, feel, and behavior of a natural breast.

The next evolution of lightweight breast prostheses related to dual chambered products. In U.S. Pat. No. 5,902,335, issued to the present inventor, a dual chambered, light weight breast prosthesis was disclosed that reduced the overall weight of an equivalent single chambered prosthesis containing only silicone gel by approximately 10 to 20%. Snyder disclosed a prosthesis with an outer chamber comprised of standard silicone gel, with an inner chamber composed of a lightweight mixture of silicone gel admixed with glass microspheres. This advancement was clearly a step forward in the production of a lighter-weight breast prosthesis, particularly for the single breast mastectomy patient. However, a pressing need developed for an even lighter weight breast prosthesis.

In a single breast mastectomy patient, the aforementioned lightweight version works well to give the patient the look and feel of a natural breast, while lessening back strain and fatigue. This particular weight ratio also retains a fairly close match to the patient's remaining breast to retain proper balance and strain on the spine. However, the double, or bilateral breast mastectomy patient has no such need for the moderate weight of the prosthesis; hence, the demand for the lightest prosthesis possible that retains the look, feel, and behavior of a natural breast has arisen.

In view of the limitations of products currently known in the art, a tremendous need exists for an extremely lightweight breast prosthesis, which retains the look, feel, and behavior of a natural breast. Applicant's invention, by its novel design provides a solution in view of currently available devices.

### SUMMARY OF THE INVENTION

In view of the foregoing, it is an object of the present invention to provide an external breast prosthesis that is exceptionally lightweight.

It is another object of the present invention to provide an external breast prosthesis that closely mimic's the look of a natural breast.

It is another object of the present invention to provide an external breast prosthesis that closely mimic's the feel of a natural breast.

It is another object of the present invention to provide an external breast prosthesis that closely mimic's the behavior of a natural breast.

It is another object of the present invention to provide an external breast prosthesis that is comfortable to wear.

It is another object of the present invention to provide an external breast prosthesis that will minimize the strain on the user's spine.

In satisfaction of these and other related objectives, the present invention provides an external breast prosthesis that is 40 to 50% lighter than a prosthesis made entirely of silicone gel. The present invention further provides a dual chambered breast prosthesis with a third extremely lightweight layer having a semi-adhesive surface to allow the prosthesis to act and behave as one unit. As will be discussed in the specification to follow, practice of the present invention provides the bilateral breast mastectomy patient an extremely lightweight option with the natural look and feel needed to wear the prosthesis throughout her waking hours while limiting the strain translated to her spine.

The preferred embodiment of the present invention incorporates an outer section extending from the back of the prosthesis and being disposed over a first inner section and a lightweight, removable innermost section. The outer section contains a reduced density gel; preferably the outer section is comprised of silicone gel admixed with a density reducing agent to form a lightweight silicone gel mixture. The density reducing agent can be any of a variety of materials with a density less than that of silicone gel, such as entrapped air or gas bubbles, expanded perlite, polystyrene beads, glass microspheres, or plastic microspheres; however, in the preferred embodiment of the present invention, the outer section is filled with a mixture of silicone and glass microspheres. This, of course results in a lighter weight material section than could be produced from the silicone gel alone. Additionally, in the preferred embodiment, the outer section mixture gives the prosthesis a more natural look and softer feel than that of regular silicone gel.

The first inner section contains a thin layer of gel that is firmer and denser than the outer section. A number of gel-like materials may suffice; however, the preferred embodiment of the present invention contains standard weight silicone gel. This inner section lines the inner portion of the softer, lighter outer section to give an added dimension of support that provides a more natural shape and behavioral characteristics to the prosthesis.

Finally, the innermost section of the present invention is comprised of a durable, extremely lightweight foam material. The foam material is backed by a soft fabric material. The opposing side of the foam insert is coated with a semi-adhesive material to give it a tacky or sticky feel. This tacky feel allows the innermost section to “grab” the outer material of the other two sections, which results in a prosthesis that moves and acts as one unit, but remains a separate section that can be removed and replaced as desired.

As will be obvious from the specification to follow, the three sections may be varied and manipulated a number of ways in order to achieve the desired balance between weight reduction and the look, feel and behavior of the overall prosthesis. However, the Applicant has found that a 40 to 50% reduction in weight can be achieved while retaining a natural look and feel of the prosthesis by manipulating the size and weight of each of the sections such that the outer section is between 71% and 80% of the entire weight of the prosthesis; the inner section is 11% to 16% of the entire weight of the prosthesis; and the innermost section is 7% to 13% of the entire weight of the prosthesis.

In summary, then, an embodiment of the present invention provides an extremely lightweight, external breast prosthesis, while retaining the look, feel, and behavior of a natural breast.

### BRIEF DESCRIPTION OF THE DRAWINGS

Applicant's invention may be further understood from a description of the accompanying drawings, wherein unless otherwise specified, like referenced numerals are intended to depict like components in the various views.

FIG. 1 is a cross-sectional view of the present invention fully assembled.

FIG. 2 is a cross-sectional view of the outer sections of the present invention.

FIG. 3 is a cross-sectional view of the innermost section of the present invention.

### DETAILED DESCRIPTION OF THE PREFERRED EMBODIMENT

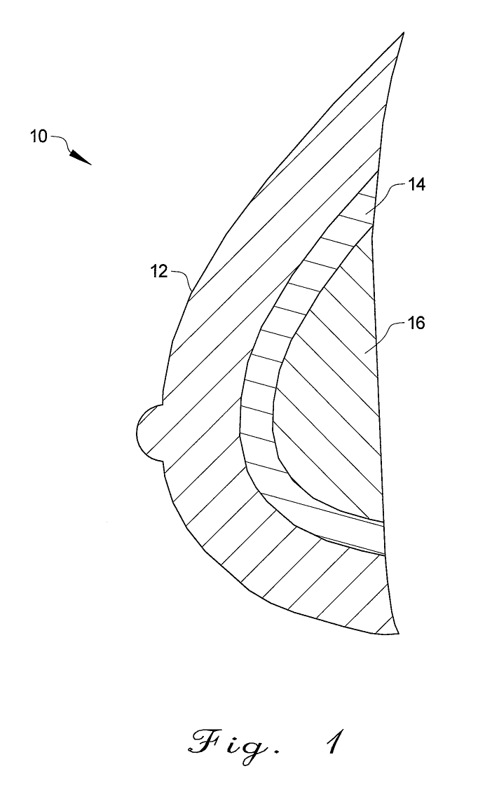

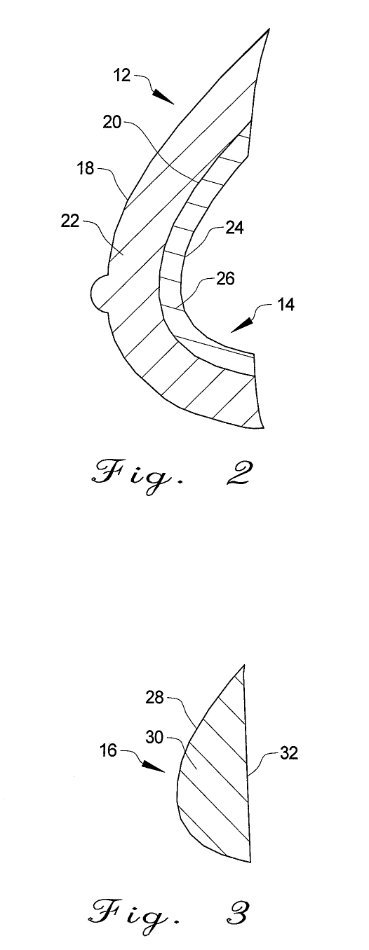

Referring to FIG. 1, a lightweight, external breast prosthesis is shown and is generally designated by numeral 10. In the preferred embodiment, the primary components incorporated by the present invention are outer section (12), inner section (14), and innermost section (16). As best seen in FIG. 2, outer section (12) is comprised of an outer surface (18) adhered or bonded to middle surface (20) and filled with density reduced gel mixture (22). Correspondingly, inner section (14) is comprised of middle surface (20) adhered or bonded to inner surface (24) and filled with standard gel (26). Finally, as best seen in FIG. 3, innermost section (16) is comprised of coating (28), lightweight form (30), and backing material (32).

In the preferred embodiment, outer surface (18), middle surface (20), and inner surface (24) are composed of thin sheets of a flexible material such as polyurethane. Outer surface (18) is first formed into a desired size of a natural, human breast shape in one of a variety of methods as known in the art. Correspondingly, middle surface (20) and inner surface (24) are adhered or bonded to outer surface (18) using a standard method as known in the art as well. Next, the envelope formed by the engagement of outer surface (18) and middle surface (20) is filled with density reduced gel mixture (22). Finally, the envelope formed by the engagement of middle surface (20) and inner surface (24) is filled with standard gel (26). Then, outer section (12) and inner section (14) are cured as known in the art.

In the preferred embodiment, standard gel (26) is comprised of a standard silicone gel with properties that allow the mimicking of natural breast tissue. Various types of silicone gel are well known for use in breast prostheses, both external and implants, and any of these gels is suitable in the practice of the present invention. However, standard gel (26), in the preferred embodiment of the present invention is comprised of a silicone gel with an approximate density of 0.97 cc.

The composition of density reduced gel mixture (22) is comprised of a standard gel admixed with a density reducing agent, i.e. an agent having a lower density than the standard gel itself for desired weight reduction. The density reducing agent may be comprised of any of a number of density reducing materials, including entrapped air or gas bubbles, expanded perlite, polystyrene beads, glass microspheres, or plastic microspheres. In the preferred embodiment of the present invention, gel mixture (22) is comprised of a standard silicone gel (similar to gel (26)) admixed with glass microspheres. More particularly, in the preferred embodiment of the present invention, density reduced gel mixture (22) has a density of approximately 70 to 80% of that of standard gel (26).

Referring back to FIG. 3, innermost section (16) is comprised of coating (28), lightweight form (30), and backing material (32). Lightweight form (30) is comprised of a lightweight material with extremely high forming properties and a correspondingly extremely low density. In the preferred embodiment, lightweight form (30) is comprised of a lightweight foam material. In the manufacturing process, the foam material is mixed and poured into a mold where backing material (32) is applied. Backing material (32) can be one of various, cloth-type materials as known in the art; however, in the preferred embodiment, backing material (32) is comprised of a cotton fabric for maximum comfort and breathability. After the foam molds, coating (28) is applied to the surface of the foam, and innermost section (16) is heated and cured to give the surface of innermost section (16) a sticky or tacky feel, which allows innermost section (16) to grab inner section (14) when inserted, and it allows prosthesis (10) to move and function as one unit. In the preferred embodiment, coating (28) is a silicone elastomer based material; however, other substances are contemplated, which may be substituted. The necessary property of coating (28) being the sticky or tacky feel upon finalization of innermost section (16).

In summary, it is imperative to the understanding of the present invention that prosthesis (10) be formulated such that outer section (12) be the largest section by percentage of weight; however, the proportions of sections (12,14,16) may be otherwise varied and manipulated to achieve the desired balance between the weight characteristic and the look, feel and behavior of prosthesis (10) itself. Correspondingly, in the preferred embodiment of the present invention, Applicant has found that the most desired balance can be achieved by proportioning the sections in the following manner: outer section (12) comprises 71 to 80% of the weight of prosthesis (10); inner section (14) comprises 11 to 16% of the weight of prosthesis (10); and innermost section (16) comprises 7 to 13% of the weight of prosthesis (10). When prosthesis (10) is produced in the aforementioned range, it has been found that the present invention achieves a 40 to 50% weight reduction from that of a prosthesis solely containing standard silicone gel. Moreover, the preferred range allows the present invention to retain the look, feel, and behavior of a natural breast.

Although the invention has been described with reference to specific embodiments, this description is not meant to be construed in a limited sense. Various modifications of the disclosed embodiments, as well as alternative embodiments of the inventions will become apparent to persons skilled in the art upon reference to the description of the invention. It is, therefore, contemplated that the appended claims will cover such modifications that fall within the scope of the invention.

I claim:

1. A breast prosthesis, comprising:
a first section, said first section comprising an outer layer of said breast prosthesis;
a second section, said second section comprising an inner layer of said breast prosthesis; and
a third section, said third section comprising an innermost layer of said breast prosthesis.

2. The breast prosthesis of claim 1 wherein said first section comprises between 71% and 80% of the total weight of said prosthesis, said second section comprises between 11% and 16% of the total weight of said prosthesis, and said third section comprises between 7% and 13% of the total weight of said prosthesis.

3. The breast prosthesis of claim 1 wherein said first section is comprised of a reduced density gel-like substance.

4. The breast prosthesis of claim 3 wherein said second section is comprised of a gel-like substance.

5. The breast prosthesis of claim 4 wherein said third section is comprised of a low density, formable material.

6. The breast prosthesis of claim 5 wherein said reduced density gel-like substance is silicone gel admixed with a density reducing agent.

7. The breast prosthesis of claim 6 wherein said density reducing agent is a plurality of glass microspheres.

8. The breast prosthesis of claim 7 wherein said gel-like substance is silicone gel.

9. The breast prosthesis of claim 8 wherein said low density formable material is foam.

10. The breast prosthesis of claim 9 wherein said first section comprises between 71% and 80% of the total weight of said prosthesis, said second section comprises between 11% and 16% of the total weight of said prosthesis, and said third section comprises between 7% and 13% of the total weight of said prosthesis.

11. A breast prosthesis, comprising:
a first section, said first section having a first, outer layer adhered to a first inner layer, wherein said envelope of said first outer layer and said first inner layer contains a reduced density gel, said first section being formed in the shape of a human breast;
a second section, said second section being comprised of said first inner layer adhered to a second inner layer, wherein said envelope of said first inner layer and said second inner layer contains a standard density gel, said second section being substantially thinner than said first section; and
a third section, said third section being comprised of a low density, formable foam material having a cloth-like backing on a first side and a coating on a second side, said second side being configured for mating with said second section, said coating providing some adherence between said second and said third section.

12. The breast prosthesis of claim 11 wherein said reduced density gel has a density that is between 70% and 80% of the density of said standard density gel.

13. The breast prosthesis of claim 12 wherein said third section is removable.

14. The breast prosthesis of claim 13 wherein said coating is a silicone elastomer-based material.

15. The breast prosthesis of claim 14 wherein said first section comprises between 71% and 80% of the total weight of said prosthesis, said second section comprises between 11% and 16% of the total weight of said prosthesis, and said third section comprises between 7% and 13% of the total weight of said prosthesis.

16. A breast prosthesis, comprising:
a first section, said first section having a first, outer layer adhered to a first inner layer, wherein said envelope of said first outer layer and said first inner layer contains a reduced density gel, said first section being formed in the shape of a human breast, said first section comprising between 71% and 80% of the total weight of said breast prosthesis;
a second section, said second section being comprised of said first inner layer adhered to a second inner layer, wherein said envelope of said first inner layer and said second inner layer contains a standard density gel, said second section being substantially thinner than said first section, said second section comprising between 11% and 16% of the total weight of said breast prosthesis; and
a third section, said third section being comprised of a low density, formable foam material having a cloth-like backing on a first side and a coating on a second side, said second side being configured for mating with said second section, said coating providing some adherence between said second and said third section, said third section comprising between 7% and 13% of the total weight of said breast prosthesis.
